(19) United States
(12) Patent Application Publication
(10) Pub. No.: US 2016/0242632 A1
(43) Pub. Date: Aug. 25, 2016

(54) System and Method for Capsule Device with Multiple Phases of Density

(71) Applicants: Ganyu LU, Palo Alto, CA (US); Kang-Hual WANG, Saratoga, CA (US); Gordon C. wilson, San Francisco, CA (US); CAPSO VISION, INC., Saratoga, CA (US)

(72) Inventors: Ganyu Lu, Palo Alto, CA (US); Kang-Huai Wang, Saratoga, CA (US); Gordon C. Wilson, San Francisco, CA (US)

(21) Appl. No.: 15/025,552

(22) PCT Filed: Oct. 22, 2013
(86) PCT No.: PCT/US13/66011

§ 371 Date: Mar. 29, 2016

(51) Int. Cl. A61B 1/00; A61B 1/06; A61B 1/273; A61B 5/00; A61B 5/145; A61B 5/03; A61B 5/11; A61B 1/04; A61B 1/31

(52) U.S. Cl. CPC A61B 1/00156; A61B 1/041; A61B 1/0661; A61B 1/0002; A61B 1/2736; A61B 1/31; A61B 1/00006; A61B 5/14539; A61B 5/037; A61B 5/1128; A61B 5/14546; A61B 5/6861; A61B 1/00124; A61B 1/00013; A61B 1/00032

(57) ABSTRACT

A capsule device with density control is disclosed, where the density of the capsule device is set to desired specific gravities when it travels to designated regions of the gastrointestinal track. The density control means will cause at least two specific gravities of the capsule device for at least two designated regions of the gastrointestinal track respectively, wherein each of said at least two specific gravities is selected from a first group consisting of a greater-than-one state and a less-than-one state. In one embodiment, the greater-than-one state correspond to the specific gravity of about 1.1 or larger and the less-than-one state corresponds to the specific gravity of about 0.94 or smaller.

### CROSS REFERENCE TO RELATED APPLICATIONS

The present invention is related to U.S. Pat. No. 7,983,458, entitled “in vivo Autonomous Camera with On-Board Data Storage or Digital Wireless Transmission in Regulatory Approved Band”, granted on Jul. 19, 2011, PCT Patent Application Series No. PCT/US13/39317, entitled “Optical Wireless Docking System for Capsule Camera”, filed on May 2, 2013 and PCT Patent Application Series No. PCT/US13/42490, entitled “Capsule Endoscopic Docking System”, filed on May 23, 2013. The U.S. patent and PCT Patent Applications are hereby incorporated by reference in their entireties.

### FIELD OF THE INVENTION

The present invention relates to diagnostic imaging inside the human body. In particular, the present invention relates to a capsule device with density control so that the capsule device has specific gravity greater than one in some regions of the gastrointestinal track and has densities less than one in other regions of the gastrointestinal track.

### BACKGROUND AND RELATED ART

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS array, and transfer the image data as an electrical signal to the proximal end through a cable. Endoscopes allow a physician control over the field of view and are well-accepted diagnostic tools. However, they do have a number of limitations, present risks to the patient, are invasive and uncomfortable for the patient, and their cost restricts their application as routine health-screening tools.

Because of the difficulty traversing a convoluted passage, endoscopes cannot easily reach the majority of the small intestine and special techniques and precautions, that add cost, are required to reach the entirety of the colon. Endoscopic risks include the possible perforation of the bodily organs traversed and complications arising from anesthesia. Moreover, a trade-off must be made between patient pain during the procedure and the health risks and post-procedural down time associated with anesthesia.

An alternative in vivo image sensor that addresses many of these problems is the capsule endoscope. A camera is housed in a swallowable capsule, along with a radio transmitter for transmitting data, primarily comprising images recorded by the digital camera, to a base-station receiver or transceiver and data recorder outside the body. The capsule may also include a radio receiver for receiving instructions or other data from a base-station transmitter. Instead of radio-frequency transmission, lower-frequency electromagnetic signals may be used. Power may be supplied inductively from an external inductor to an internal inductor within the capsule or from a battery within the capsule.

An autonomous capsule camera system with on-board data storage was disclosed in the U.S. Pat. No. 7,983,458, entitled “In Vivo Autonomous Camera with On-Board Data Storage or Digital Wireless Transmission in Regulatory Approved Band,” granted on Jul. 19, 2011. This patent describes a capsule system using on-board storage such as semiconductor nonvolatile archival memory to store captured images. After the capsule passes from the body, it is retrieved. Capsule housing is opened and the images stored are transferred to a computer workstation for storage and analysis. For capsule images either received through wireless transmission or retrieved from on-board storage, the images will have to be displayed and examined by diagnostician to identify potential anomalies.

FIG. 1 illustrates an exemplary capsule system with on-board storage. The capsule system 110 includes illuminating system 12 and a camera that includes optical system 14 and image sensor 16. A semiconductor nonvolatile archival memory 20 may be provided to allow the images to be stored and later retrieved at a docking station outside the body, after the capsule is recovered. System 110 includes battery power supply 24 and an output port 26. Capsule system 110 may be propelled through the GI tract by peristalsis.

Illuminating system 12 may be implemented by LEDs. In FIG. 1, the LEDs are located adjacent to the camera's aperture, although other configurations are possible. The light source may also be provided, for example, behind the aperture. Other light sources, such as laser diodes, may also be used. Alternatively, white light sources or a combination of two or more narrow-wavelength-band sources may also be used. White LEDs are available that may include a blue LED or a violet LED, along with phosphorescent materials that are excited by the LED light to emit light at longer wavelengths. The portion of capsule housing 10 that allows light to pass through may be made from bio-compatible glass or polymer.

Optical system 14, which may include multiple refractive, diffractive, or reflective lens elements, provides an image of the lumen walls on image sensor 16. Image sensor 16 may be provided by charged-coupled devices (CCD) or complementary metal-oxide-semiconductor (CMOS) type devices that convert the received light intensities into corresponding electrical signals. Image sensor 16 may have a monochromatic response or include a color filter array such that a color image may be captured (e.g. using the RGB or CYM representations). The analog signals from image sensor 16 are preferably converted into digital form to allow processing in digital form. Such conversion may be accomplished using an analog-to-digital (AD)) converter, which may be provided inside the sensor (as in the current case), or in another portion inside capsule housing 10. The A/D unit may be provided between image sensor 16 and the rest of the system. LEDs in illuminating system 12 are synchronized with the operations of image sensor 16. Processing module 22 may be used to provide processing required for the system such as image processing and video compression. The processing module may also provide needed system control such as to control the LEDs during image capture operation. The processing module may also be responsible for other functions such as managing image capture and coordinating image retrieval.

After the capsule camera traveled through the GI tract and exits from the body, the capsule camera is retrieved and the images stored in the archival memory are read out through the output port. The received images are usually transferred to a base station for processing and for a diagnostician to examine. The accuracy as well as efficiency of diagnostics is most important. A diagnostician is expected to examine all images and correctly identify all anomalies.

When the capsule device travels through the gastrointestinal (GI) track, the capsule device will encounter different environments. It is desirable to manage the capsule device to travel at a relatively steady speed so that sensor data (e.g., images) sufficient data is collected at all locations along the portion of the GI tract which is of interest, without wasting power and data storage collecting excessive data in some locations. In some environments, it is desirable to have a heavier capsule density. In other environments, it may be desirable to have a lighter capsule density. Capsule endoscopy is typically performed with ambulatory patients whose torsos are erect a majority of the time. The transit of the capsule is hastened if the capsule is denser than its surrounding fluid when it must move down in the direction of gravity and less dense when it must move up against gravity. For example, when the capsule is in the stomach where the stomach is filled with liquid, the capsule will float above the liquid if the capsule has a lighter density than the liquid. In this case, it would be hard for the capsule device to get to the small intestine. Therefore, it is desirable for the capsule device to have a heavier density than the liquid when the capsule device is in the stomach. On the other hand, when the capsule passes through small bowel and enters the cecum, it has to transit through the ascending colon. If the capsule device has a heavier density than the liquid in the ascending colon, it would take a long time for the capsule device to travel through the ascending colon. Therefore, it is desirable for the capsule device to have a lighter density than the liquid when the capsule device is in the ascending colon. Furthermore, when the capsule is in the descending colon, it is desirable that it have a density greater than that of the liquid in the colon. Therefore, it is desirable to be able to control the capsule density to allow the capsule to have different densities in different sections of the GI track so that the capsule device will travel through the GI track at a proper pace.

### BRIEF SUMMARY OF THE INVENTION

The present invention discloses a capsule device with density control so that the capsule device has desired specific gravities when it travels to designated regions in the gastrointestinal track. The capsule device comprises a sensor system and a density control means. The sensor system may include a light source, an image sensor for capturing image frames of a scene illuminated by the light source, an archival memory, and a housing. The housing can be adapted to be swallowed. The light source, the image sensor and the archival memory are enclosed in the housing. The density control means will cause at least two specific gravities of the capsule device for at least two designated regions of the gastrointestinal track respectively, wherein each of said at least two specific gravities is selected from a first group consisting of a greater-than-one state and a less-than-one state. In one embodiment, the greater-than-one state correspond to the specific gravity of about 1.1 or larger and the less-than-one state corresponds to the specific gravity of about 0.94 or smaller.

In one embodiment, said at least two designated regions of the gastrointestinal track can be selected from a second group comprising stomach, ascending colon and descending colon. Said at least two designated regions of the gastrointestinal track may correspond to stomach and ascending colon, and the corresponding said at least two specific gravities are the greater-than-one state and the less-than-one state respectively. In another embodiment, said at least two designated regions of the gastrointestinal track correspond to stomach, ascending colon and descending colon, and wherein the corresponding said at least two specific gravities are the greater-than-one state, the less-than-one state and the greater-than-one state respectively.

In order to configure the capsule device with a proper specific gravity, the region of the gastrointestinal track where the capsule device is located needs to be determined. The region can be determined based on estimated transit time after the capsule device is swallowed, pH values or luminal pressure measured by the capsule device, identification of image contents based on captured images by the capsule device, motion detection based on the captured images by the capsule device or colonic microflora detected at the capsule device location according to embodiments of the present invention.

In another embodiment, said density control means couples a deformable member to the sensor system, wherein the deformable member contains gas generating material, said density control means causes the deformable member to inflate by causing gastric fluid to enter the deformable member so that the gas generating material generates gas and the capsule device has the specific gravity less than one. The deformable member can be coated with biodegradable coating to prevent the gastric fluid to enter the deformable member before the capsule device exits stomach. The deformable member can be made of a first material, wherein the first material is permeable to gastric fluid and the first material is less permeable to the gas than the gastric fluid. After a period of time since the capsule device reaches the specific gravity less than one, the density control means may cause the capsule device reach the specific gravity greater than one by allowing the gastric fluid to continue to enter the deformable member and the gas to continue to leak from the deformable member.

The capsule device may have electrical contacts fixedly disposed on the housing, wherein the electrical contacts are coupled to the archival memory so that an external device is allowed to access image data stored in the archival memory through the electrical contacts. The electrical contacts may include power pins to provide power to the capsule device for data retrieval of image data stored on the archival memory. Alternatively, inductive powering can be used to provide power to the capsule device for data retrieval of image data stored on the archival memory. In yet another embodiment, the capsule device further comprises an optical transmitter to transmit an optical signal through a clear window, wherein image data from the archival memory is transmitted to an external optical receiver.

### BRIEF DESCRIPTION OF THE DRAWINGS

FIG. 1 shows schematically a capsule camera system in the GI tract, where archival memory is used to store captured images to be analyzed and/or examined.

FIG. 2A-FIG. 2E illustrate an example of various density states for a capsule device incorporating density control according to an embodiment of the present invention.

FIG. 3A-FIG. 3B illustrate an example of various density states for a capsule device incorporating a biodegradable plug according to an embodiment of the present invention.

FIG. 4 illustrates an example of a capsule device incorporating density control according to an embodiment of the present invention, where the housing includes a flexible section to expand or contract.

FIG. 5A and FIG. 5B illustrate an example of a capsule device incorporating density control according to an embodiment of the present invention, where the housing comprises two closely coupled parts.

FIG. 6 illustrates an example of a capsule device incorporating density control according to an embodiment of the present invention, where an extendable part is attached to the sensor system.

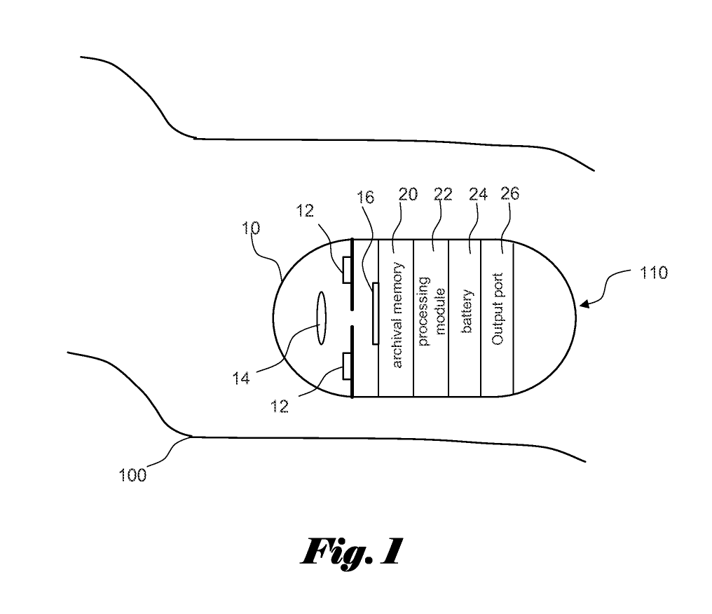

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely representative of selected embodiments of the invention.

Reference throughout this specification to “one embodiment,” “an embodiment,” or similar language means that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention.

The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

In U.S. Pat. No. 7,192,397 and U.S. Pat. No. 8,444,554, a capsule device with specific gravity about 1 is disclosed. When the capsule device has a specific gravity about 1, the device will suspend or float in the liquid in the gastrointestinal (GI) track such as in the stomach or in the colon. As disclosed in U.S. Pat. No. 7,192,397 and U.S. Pat. No. 8,444,554, the capsule device will be carried through the body lumen by a flow of liquid through the body lumen when the capsule device has a specific gravity about 1. However, for an in vivo capsule device, after the capsule device is swallowed by a patient, the capsule device first goes through the pharynx and esophagus into the stomach and the stomach may be filled with liquid. If the specific gravity of the capsule device is less than 1 or the capsule device has a lighter density than the liquid, it will float on the surface of the liquid inside stomach. Thus, it is not conducive for the capsule device to transit through the pylorus into the small bowel.

For a capsule device with an image sensor, it is critical to have a steady and consistent travelling velocity inside different regions of the GI tract. e.g. stomach, small bowel, ascending and descending colons so that smooth and stable images and video can be obtained. The travelling velocity of the capsule camera depends on many factors including regional gastrointestinal motility, gravitational force, buoyancy and viscous drag of the surrounding fluids. After the capsule device is swallowed, it is propelled into the esophagus. Peristaltic waves in the esophagus move the camera into the stomach. After the capsule device passes the cardia and enters the stomach with fluid, the balance among gravitational force, buoyancy and drag from the gastric fluids starts to affect its travelling velocity and transit time. The migrating myoelectric cycle (MMC) can be divided into four phases. Phase 1 lasts between 30 and 60 minutes with rare contractions. Phase 2 lasts between 20 and 40 minutes with intermittent contraction. Phase 3, or housekeeping phase, lasts between 10 and 20 minutes with intense and regular contractions for short period. The housekeeping wave sweeps all the undigested material out of the stomach to the small bowel. Phase 4 lasts between 0 and 5 minutes and occurs between phase 3 and phase 1 of two consecutive cycles. For the capsule device to travel aborally at a desired velocity in all four phases, preferably phases 1 and 2, its specific gravity needs to be greater than 1 (e.g., 1.1) to overcome the buoyance and drag from the surrounding fluid. If phase 3 is detected through image motion detection or accelerometer, the specific gravity can be pushed to a value less than one (e.g., 0.95) for the capsule device to float to the top and to retake the video in a more stable phases.

In the small intestine, BER (basic electrical rhythm) is around 12 cycles/minute in the proximal jejunum and decreases to 8 cycles/minutes in the distal ileum. There are three types of smooth muscle contractions: peristaltic waves, segmentation contractions and tonic contractions. Normally, peristalsis will propel the capsule device towards large intestines. Since the small intestine twists and turns around between the stomach and the large intestine, the capsule device may sometimes be trapped at corners and turns. In this case, motion detection may be used to detect such situation. Accordingly, density-changing mechanisms can be used to slightly change the balance between gravity and buoyancy so that the capsule device can leave the trap sooner before the next peristalsis.

While the large intestine is one organ, it demonstrates regional differences. The proximal (ascending) colon serves as a reservoir and the distal (transverse and descending) colon mainly performs as a conduit. The character of the luminal contents impacts the transit time. Liquid passes through the ascending colon quickly, but remains within the transverse colon for a long period of time. In contrast, a solid meal is retained by the cecum and ascending colon for longer periods than a liquid diet. In the ascending colon, retrograde movements are normal and occur frequently. In order for the buoyant force to overcome the gravitational force and retropulsion, the specific gravity of the capsule device according to an embodiment of the present invention is decreased to less than less than one (e.g., 0.94 or less) after the capsule enters the large intestine. Alternatively, the density of the capsule device as a whole has lighter density than the surrounding fluid. In the descending colon and rectum, propulsive contractions prevail. The capsule device is carried aborally towards the rectum by the natural propulsion. However, increasing the specific gravity of the apparatus to larger than one (e.g., 1.1 or larger) can shorten the transit time and allow a smooth and steady motion.

In order to properly set the specific gravity or the density of the capsule device, it needs to know which regions of the GI track that the capsule device is located. There are various know region detection methods in the literature. The region detection methods include estimated transit time (e.g., about 1 hour in stomach and about 3-4 hours in small bowel), identification of image contents based on captured images by the capsule device, motion detection based on the captured images by the capsule device, pH detection (pH value increasing progressively from the stomach (1.5-3.5) and the small bowel (5.5-6.8) to the colon (6.4-7.0), pressure sensor (higher luminal pressure from peristaltic motion in the colon than that in the small bowel) and colonic microflora. The ascending colon has a larger diameter than other regions besides the stomach. The size may be detected by the methods disclosed in U.S. Patent Publications, Series No. 2007/0255098, published on Nov. 1, 2007, U.S. Patent Publications, Series No. 2008/0033247 published on Feb. 7, 2008 and U.S. Patent Publications, Series No. 2007/0249900, published on Oct. 25, 2007.

According to one embodiment of the present invention, the capsule device is configured to have a specific gravity (SG) larger than 1 or a density higher than the liquid in the stomach when the capsule device is in the stomach. For example, the capsule device is made such that its specific gravity is equal to 1.1 or larger when the capsule device is in the stomach. After the capsule passes through the small bowel and enters the cecum, it has to transit through the ascending colon. In this case, if the specific gravity were larger than 1 or the density of the capsule device is heavier than the density of the liquid therein, it would take a long time for the capsule to go through the ascending colon. The procedure time should not unnecessarily be prolonged so that patient does not need to fast for too long. Furthermore, the battery life for the capsule device is limited. If the capsule device stays in the ascending colon for too long, the battery may be exhausted before the capsule device finishes its intended tasks, such as capturing images of the colon. Therefore, it is preferred that the capsule device has a specific gravity less than 1 or has a lighter density than the liquid in the cecum and ascending colon. For example, the capsule device is configured so that it has density of 0.94 or less. It is contrary to the case for the stomach, where the capsule device is configured to have a specific gravity larger than one or heavier density than the liquid in the stomach.

As mentioned above, the capsule device according to one embodiment of the present invention has a density heavier than the body lumen liquid in one region of the GI tract (e.g. the stomach) and then has a density lighter than the body lumen liquid in another region of the GI track (e.g., the cecum or ascending colon). In another embodiment, the capsule device evolves into a first state with a specific gravity greater than 1 or with a density heavier than the liquid in one region of the GI track when the capsule device is in the stomach; the capsule device then evolves into a second state with a specific gravity less than 1 or with a density lighter than the liquid when the capsule device enters the ascending colon; and the capsule device further evolves into a third state with a specific gravity greater than 1 or with a density heavier than the liquid when it reaches the descending colon to assist its movement toward distal colon and sigmoid. Finally the capsule device will reach anus for excretion. A specific gravity of 1.1 or larger can be selected if the specific gravity greater than 1 is desired. A specific gravity of 0.94 or smaller can be selected if the specific gravity less than 1 is desired.

In the example described above, the capsule device will have three different density states (or specific gravity states), i.e., starting with a high density (SG≧1.1), transitioning to a low density (SG≦0.94), and back to high density (SG≧1.1). However, the present invention is also applicable to other multiple density states having two or more different states. While a high density with SG≧1.1 and a low density with SG≦0.94 are used as an example, other high density range and other low density range can also be used to practice the present invention.

In order to achieve a desired density or specific gravity, the capsule device may comprise a deformable member that can transform from a collapsed state to an expanded state. In one embodiment, the deformable member that can transform from a collapsed state to an expanded state is returnable to at least partially collapsed state after an extended period of time, such as several hours. The deformable member can be coated with enteric coating, which will remain intact in stomach and other areas of the GI track with low pH values. However, the deformable member with enteric coating will dissolve when it approaches the terminal ileum or the cecum, where the pH value rises to a higher level. In one embodiment, the deformable member comprises an inflatable shell having an internal space. At least a portion of the inflatable shell is permeable to external fluid, such as water or gastric juice. The inflatable shell contains a chemical that will generate gas when the chemical is combined with water. The gas generated will inflate the deformable member to render the density of the capsule device as a whole substantially less than 1. For example, the specific gravity can be 0.94 or less, or the density of the capsule device as a whole is lighter than the density of the liquid in the environment corresponding to the distal small bowel or colon. For example, effervescent granules are known to generate gases such as CO2 when mixed with water, which can be deposited inside the deformable member. In the field, there are known materials that are permeable to water. Therefore, after an extended period of time, more liquid will enter the inflatable shell and the capsule device will return to a state with SG greater than 1 or with a density heavier than the surrounding liquid.

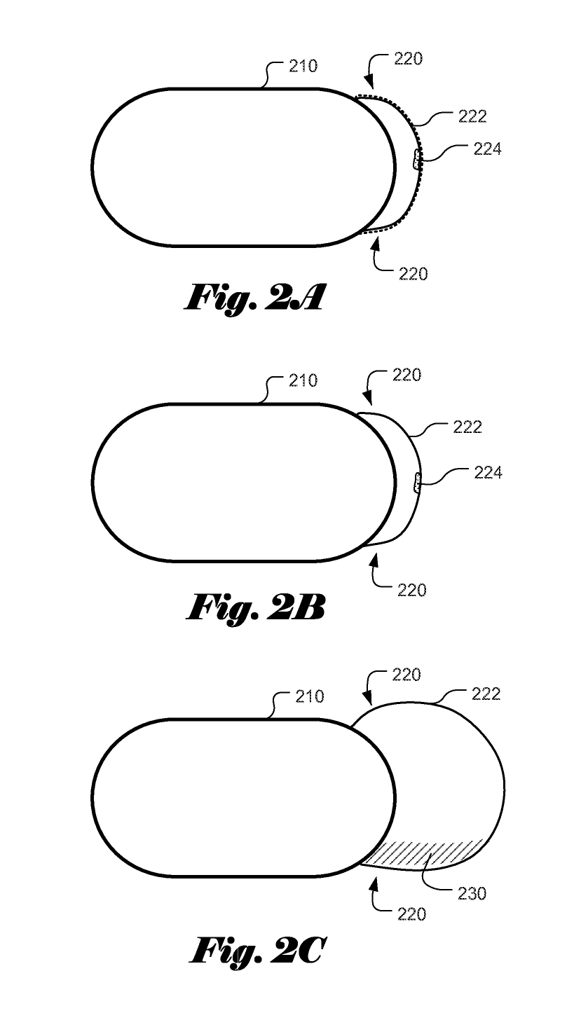

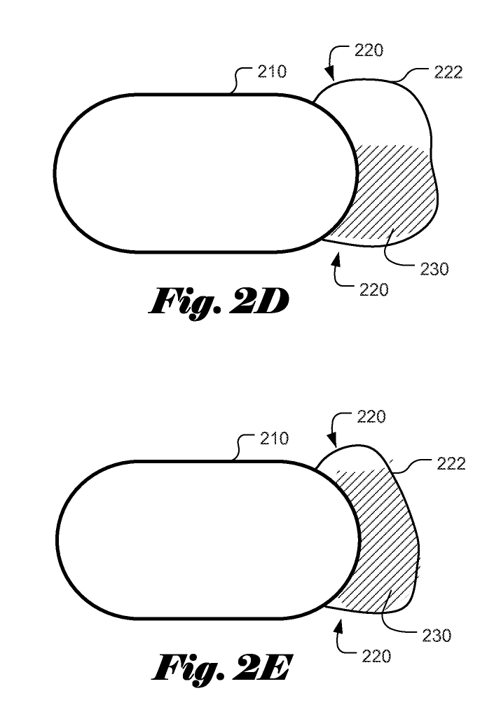

FIGS. 2A-2C illustrate an example of a capsule device with a deformable member at different states according to an embodiment of the present invention. FIG. 2A illustrates the capsule device before it is expanded. The capsule device comprises a sensing system (210) and a deformable member (220). The deformable member comprises an inflatable shell (222), which is a semipermeable membrane, containing effervescent material 224. The inflatable shell is expandable and made of material that is permeable to external fluid, such as water or gastric juice. Furthermore, an enteric coating (as shown by dashed lines) can be applied to the outer surface of the inflatable shell. The enteric coating may also cover the entire capsule system. Instead of coating the system, the capsule may be put into a capsule shell. The shell may be similar to the capsule shells used to deliver oral pharmaceuticals. These shells are designed to dissolve in the stomach or small bowel within about 30 minutes of swallowing, unless they are enteric, in which case they will not dissolve in the low pH of the stomach but disintegrate in the higher pH environment of the small bowel or colon. The shell may be made of polymers, polysaccharides, plasticizers, methyl cellulose, gelatin, sugar, or other materials. Methacrylic acid co-polymer type C is an example of an enteric polymer. These materials may also be applied as coatings to the deformable member alone or to it and the sensing system.

When the capsule device approaches the terminal ileum or the cecum, the enteric coating will dissolve due to the higher pH level, as shown in FIG. 2B. With the enteric coating dissolved, external fluid will gradually get into the deformable member. When the fluid makes contact with the effervescent material, gas will be generated to expand the deformable member as shown in FIG. 2C. While a small amount of fluid (230) gets into the deformable member, the gas generated is able to expand the deformable member so that the capsule device as a whole has a specific gravity less than one (e.g. 0.94).

The effervescent material should be in contact with the semipermeable membrane of the deformable member so that water that diffuses through the membrane will reach the effervescent material as quickly as possible. The effervescent material may be a powder or dispersion that coats a portion of the inside surface of the membrane or it might comprise granules that rest on the surface of the membrane.

An example effervescent material is a mixture of anhydrous sodium bicarbonate and citric acid. These two substance must be dissolved in water to react. The reaction does not consume water and, in fact, generates water and carbon dioxide. If the osmolality of solution inside the deformable member exceeds that outside, water will continue to diffuse into the member by osmosis until the osmolalities are equal or the internal pressure equals the osmotic pressure. The semipermeable membrane material is selected such that it is sufficiently impermeable to CO2, or whatever gas is generated internally, so that the member fills with gas and sufficient gas is retained to keep the member inflated for the period of the procedure over which buoyancy is desired. By controlling the quantity of effervescent material in the member, the total quantity of gas produced is controlled so that an excessive pressure is not produced in the member. The initial volume of the member can be fixed if the member is made from an inelastic material. If an elastic material is used, then the final volume will be a function of the pressure.

The capsule may become trapped inside the GI tract if an obstruction such as a tumor exists. Ideally the inflatable member should deflate after the period of time allotted for a normal procedure (e.g. 10 hours) If the mass rate of diffusion of CO2 is low but exceeds that of water, the member will lose gas faster than water enters and the member will deflate and shrink before the gas volume is displaced fully by water. The reduced volume of the system increases the chance that it will pass the obstruction without the need for medical interventions such as endoscopy or surgery.

The deflation of the member increases the specific gravity of the system to a value greater than 1 (e.g. 1.1). This increased specific gravity can increase the transit time through the descending colon.

Alternatively, the inflatable shell material can be carefully selected so that the material is more permeable for fluid such as water and less permeable to gas such as CO2. Therefore, over a longer period of time, the proportion of volume inside the inflatable shell which is liquid will increase relative to the gas volume. If the shell is elastic, the total volume may decrease. If it is inelastic, the gas may be compressed as the pressure increases. Some gas may also diffuse out or be released through a pressure-relief valve. Some combination of the above may happen, depending on the design. FIG. 2D illustrates an example of the state of the capsule device after a period of time beyond the state shown in FIG. 2C. Compared to the state in FIG. 2C, the state in FIG. 2D has more fluid volume and less gas volume. FIG. 2E illustrates an example of the state of the capsule device further beyond the state in FIG. 2D. The deformable member in FIG. 2E contains mostly water so that the capsule device as a whole has a specific gravity greater than 1 (e.g., 1.1) or the density of the overall capsule device is heavier than the fluid again.

The capsule device can be designed so that it will reach the state with the SG less than 1 (e.g., 0.95) or with density lighter than the external fluid when the capsule device approaches the distal small bowel or ascending colon. Furthermore, the capsule device can be designed so that it will reach the state with the SG greater than 1 (e.g., 1.1) or with density heavier than the external fluid when the capsule device reaches or approaches the descending colon.

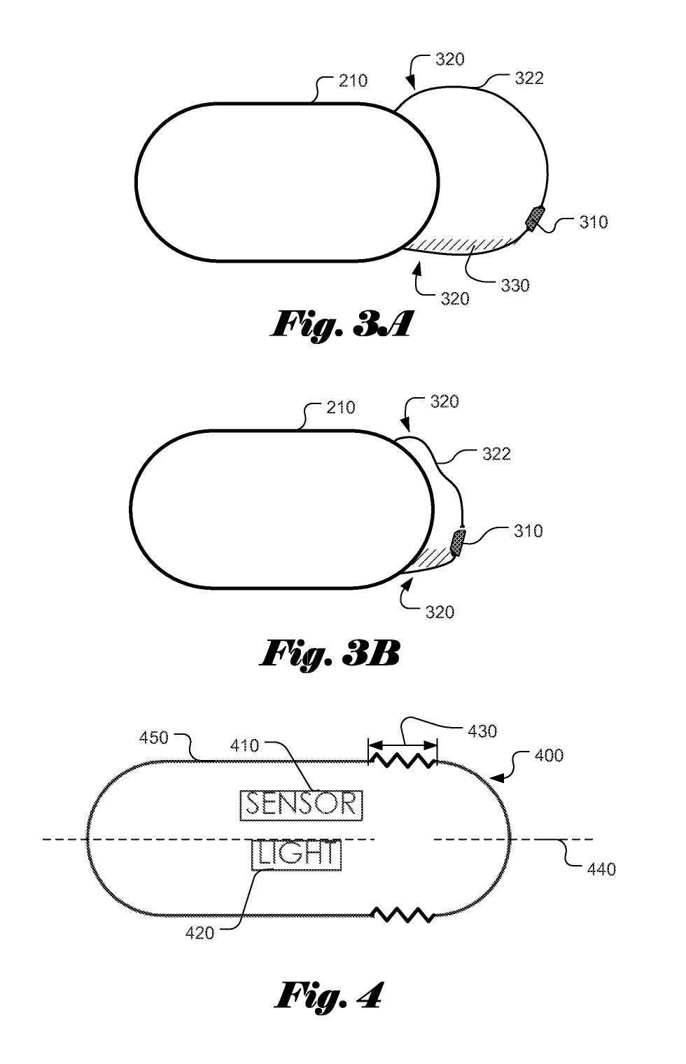

In another embodiment, the capsule device uses a different density control means to change the capsule device from a SG less than 1 to larger than 1 (or from a lighter density to a heavier density than the external fluid). In this case, the inflatable shell is made of a material substantially impermeable to CO2. A biodegradable plug is included in the deformable member as shown in FIG. 3A and FIG. 3B, where the biodegradable plug is degradable after an extended period of time, such as a few hours. As shown in FIGS. 3A-B, the capsule device according to an embodiment of the present invention includes a deformable member (320). The deformable member comprises an inflatable shell (322) and a biodegradable plug (310). The inflatable shell (322) is a semipermeable membrane, containing effervescent material (not shown in FIGS. 3A-B). FIG. 3A illustrates the state that the gas from the gas generating material causes the deformable member (322) to expand when the fluid gets into the inflatable shell. After the plug is degraded, it will be separated or partially separated from the shell, or have a gap with the shell so as to cause the gas to leak as shown in FIG. 3B. In another embodiment, at least part of the inflatable shell of the deformable member is made of a material that allows CO2 to diffuse out of the deformable member. Therefore, over an extended period of time, the capsule device density returns to higher than 1 or the density of the capsule device as a whole is heavier than the liquid in its environment. In yet another embodiment, the gas will diffuse out and the fluid will diffuse in, which will cause the capsule device density to return to higher than 1 or the density of the capsule device as a whole to be heavier than the liquid in its environment.

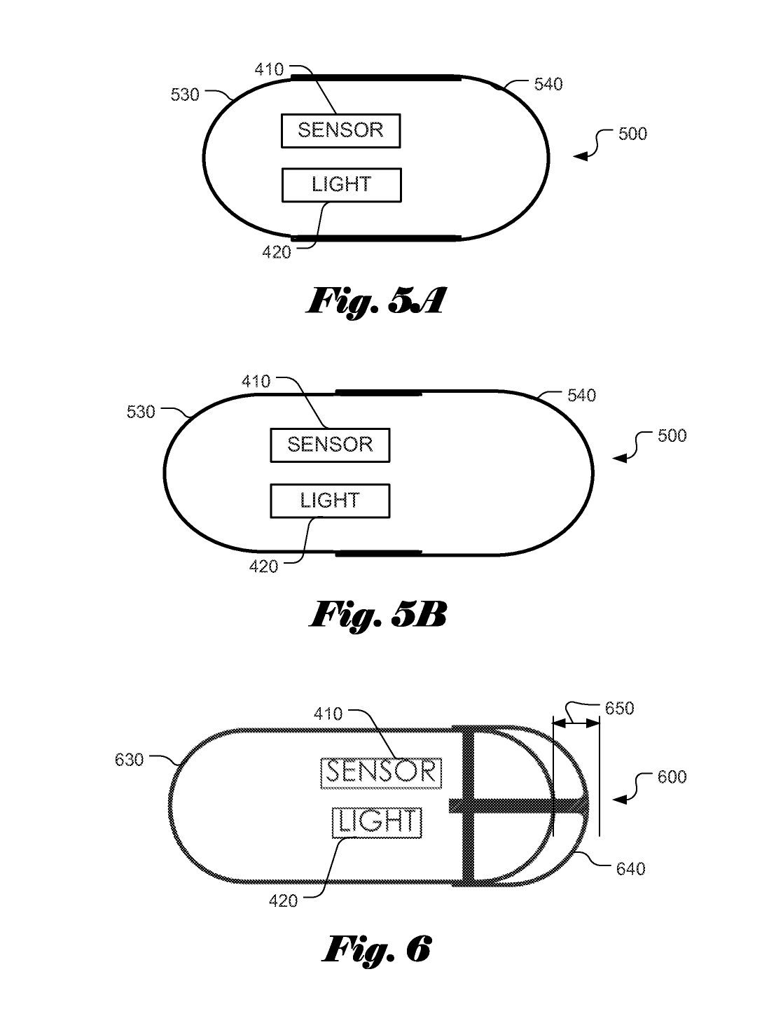

FIG. 4 illustrates another density control means, where the housing (450) of the capsule device (400) includes a flexible section (430). For example, a bellows-like structure can be used for the flexible section. The flexible section can be expanded or compressed along the longitudinal direction (440) of the capsule device. Furthermore, the capsule device in FIG. 4 comprises sensor 410 and light 420 for capturing images inside the body lumen. FIGS. 5A and 5B illustrate another expandable housing structure for a capsule device (500) incorporating density control means according to an embodiment of the present invention. The housing comprises two tightly coupled parts (530 and 540) and a locking means (not shown in FIGS. 5A and 5B) is used to prevent the two parts from being pulled apart unintentionally. While the two parts (530 and 540) form an expandable housing, the expandable housing is still maintained in a sealed condition to protect the electronic components inside the housing. The seal may be proved by an O-ring or other form of gasket or by a layer of oil or other substance which is immiscible in water and forms a moisture battier in the gap between the overlapping parts (e.g. between 530 and 540). FIG. 5A illustrates a state that the capsule device has a SG greater than 1 or has a density as a whole heavier than the liquid in the section of the GI track that the capsule device is in. FIG. 5B illustrates another exemplary density state, where the capsule device is extended to occupy volume space so that the capsule device has a SG less than 1 or has a density as a whole lighter than the liquid in the section of the GI track that the capsule device is in.

FIG. 6 illustrates yet another example of density control means, where a main sensor system (630) of a capsule device (600) is configured to accommodate an extendable attachment (640). The extendable attachment (640) can be moved within a range (650). When the extendable attachment is fully extended, the capsule device has a SG less than 1 or has a density as a whole lighter than the liquid in the section of the GI track that the capsule device is in. When the extendable attachment is fully retreated, the capsule device has a SG larger than 1 or has a density as a whole heavier than the liquid in the section of the GI track that the capsule device is in. The above examples of density control means are not meant to exhaustively list possible configurations to facilitate the density control means. A person skilled in the art may practice the present invention using similar arrangement.

The embodiments in FIGS. 4-6 illustrate capsules with expandable housings. The housings may be expanded using an actuator such as a motor and screw drive internal to the capsule. Such actuators may consume excessive power, however. Another option is to spring load the capsule internally. The capsule expansion is constrained by and external shell or coating that dissolves after the capsule is swallowed.

In another embodiment, the capsule device is coated with a material to cause the capsule slippery, i.e., having a reduced friction (comparing to case without the coating) with the body lumen or the gastric fluid. The reduced friction will allow the capsule device to travel faster under the peristalsis force so to reduce procedure time. Furthermore, slipperiness will reduce the chance that the capsule device gets trapped at corners and turns in the intestines. Hydrophilic coatings are one type of coating that increases lubricity in an aqueous medium.

In the wireless application, a transmitter is used to transmit image data to a receiver system external to the body and the image data is stored in an external recorder. In U.S. Pat. No. 5,604,531, a wireless capsule system is disclosed and the capsule system with a wireless transmitter is powered by the battery within the capsule. For colon application, the transit time is substantially longer than that for the small bowel application. Therefore, the receiver system and external recorder become burdensome to carry over long hours (e.g., 10 hours or more). Since the time period that the colon procedure takes in general is long than the span of regular office hours, it is also difficult for a patient to return the equipment in the same day. Thus, it requires leaving the equipment with the patient overnight and it is subject to equipment damage/attrition and doubles the number of equipment needed. Consequently, it will increase healthcare cost. For colon application, the transit time may be substantially longer than that for the small bowel application. Therefore, the receiver system and external recorder become burdensome to carry over long hours. It is desirable to develop a capsule device that can achieve faster transit time for the colon application.

In yet another embodiment of the present invention, the density control means is applied to a capsule system with on-board storage. A such system is disclosed in to U.S. Pat. No. 7,983,458, entitled “in vivo Autonomous Camera with On-Board Data Storage or Digital Wireless Transmission in Regulatory Approved Band”, granted on Jul. 19, 201. The capsule system with on-board storage does not require the patient to wear any external equipment. Therefore, the capsule system with on-board storage is much preferred for procedure requiring a prolonged time period. Furthermore, in PCT Patent Application No. PCT/US13/42490, a docketing station to read out archived data from a capsule system with on-board storage is disclosed. The capsule system comprises a set of probe pads disposed on the housing. After the capsule device is excreted and recovered, the image data can be retrieved by probing these probe pads without opening the capsule housing. Since the battery power is pretty much depleted when the capsule device is retrieved, one pair of the probe pads can be used to provide power and ground for the data retrieval operation. Alternatively, the power can be provided using inductive powering as disclosed in PCT Patent Application Series No. PCT/US13/39317. After the capsule is recovered, the data may be transmitted optically through a transparent portion of the capsule to an external receiver.

The invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A capsule device, comprising:
a sensor system comprising:
a light source;
an image sensor for capturing image frames of a scene illuminated by the light source;
an archival memory; and
a housing adapted to be swallowed, wherein the light source, the image sensor and the archival memory are enclosed in the housing; and
a density control means for causing at least two specific gravities of the capsule device for at least two designated regions of gastrointestinal track respectively, wherein each of said at least two specific gravities is selected from a first group consisting of a greater-than-one state and a less-than-one state.

2. The capsule device of claim 1, wherein the greater-than-one state correspond to the specific gravity of about 1.1 or larger and the less-than-one state corresponds to the specific gravity of about 0.94 or smaller.

3. The capsule device of claim 1, wherein said at least two designated regions of the gastrointestinal track are selected from a second group comprising stomach, ascending colon and descending colon.

4. The capsule device of claim 1, wherein said at least two designated regions of the gastrointestinal track correspond to stomach and ascending colon, and wherein the corresponding said at least two specific gravities are the greater-than-one state and the less-than-one state respectively.

5. The capsule device of claim 1, wherein said at least two designated regions of the gastrointestinal track correspond to stomach, ascending colon and descending colon, and wherein the corresponding said at least two specific gravities are the greater-than-one state, the less-than-one state and the greater-than-one state respectively.

6. The capsule device of claim 1, wherein whether the capsule device is located in or approaching at one of said at least two designated regions of the gastrointestinal track is determined based on:
estimated transit time after the capsule device is swallowed;
pH values measured at capsule device locations;
luminal pressure measured at the capsule device location;
identification of image contents based on captured images by the capsule device;
motion detection based on the captured images by the capsule device;
colonic microflora detected at the capsule device location; or
estimation of lumen diameter.

7. The capsule device of claim 1, wherein said density control means couples a deformable member to the sensor system, wherein the deformable member contains gas generating material, said density control means causes the deformable member to inflate by causing fluid to enter the deformable member so that the gas generating material generates gas and the capsule device has the specific gravity less than one.

8. The capsule device of claim 7, wherein the deformable member is coated with an enteric coating before the capsule device is swallowed to prevent the fluid to enter the deformable member before the capsule device exits stomach.

9. The capsule device of claim 7, wherein the deformable member comprises a biodegradable plug, wherein the biodegradable plug becomes separated or partially separated from rest of the deformable member or causes leaks on the deformable member to let the gas and the fluid to leak from the deformable member.

10. The capsule device of claim 7, wherein the deformable member is made of a first material which is more permeable to the gas than to the fluid.

11. The capsule device of claim 10, wherein the deformable member inflates with the gas and later deflates as the gas diffuses out of the deformable member faster than the fluid diffuses in the deformable member.

12. The capsule device of claim 7, wherein after a first period of time since the capsule device reaches the specific gravity less than one, the density control means causes the capsule device to reach the specific gravity greater than one by allowing the fluid to continue to enter the deformable member such that a volume ratio of the gas to the fluid inside the member decreases.

13. The capsule device of claim 1, wherein the capsule devise is coated with or made of a second material so that the capsule devise has a reduced friction with body lumen or fluid.

14. The capsule device of claim 1, wherein electrical contacts are disposed fixedly on the housing, wherein the electrical contacts are coupled to the archival memory so that an external device is allowed to access image data stored in the archival memory through the electrical contacts.

15. The capsule device of claim 14, wherein the electrical contacts include power pins to provide power to the capsule device for data retrieval of image data stored on the archival memory.

16. The capsule device of claim 14, wherein inductive powering is used to provide power to the capsule device for data retrieval of image data stored on the archival memory.

17. The capsule device of claim 1, wherein the capsule device further comprises:
an optical transmitter to transmit an optical signal through a clear window, wherein image data from the archival memory is transmitted to an external optical receiver.

18. The capsule device of claim 17, wherein inductive powering is used to provide power to the capsule device for data retrieval of image data stored on the archival memory.
